(19) United States
(12) Patent Application Publication
(10) Pub. No.: US 2005/0026297 A1
(43) Pub. Date: Feb. 3, 2005

(54) Kits and methods for preparing gell samples optmimized for dual staining

(76) Inventors: Michal Daniely, Ganey Tikva (IL); Malka Reichart, Ramat Gan (IL); Eran Kaplan, Rehovot (IL); Yulia Zilberstein, Rishon Le-Zion (IL)

Correspondence Address: Martin Moynihan;Anthony Castorina, Suite 207, 2001 Jefferson Davis Highway, Arlington, VA, 22202, US

(21) Appl. No.: 10/493,319

(22) PCT Filed:
(86) PCT No.: PCT/IL02/00894

Related U.S. Application Data
(60) Provisional application No. 60/331,061, filed on Nov. 7, 2001.

(51) Int. Cl. G01N 33/00

(52) U.S. Cl. 436/100

(57) ABSTRACT

A method of preparing nucleated peripheral blood or bone marrow cells optimized for at least dual mode imaging. The method including: (a) isolating nucleated cells from a peripheral blood or a bone marrow sample; and (b) resuspending the nucleated cells in the presence of a morphology preserver including at least 1% serum, and recovering a cell fraction thereby preparing the nucleated peripheral blood or bone marrow cells optimized for at least dual mode imaging.

### FIELD AND BACKGROUND OF THE INVENTION

The present invention relates to kits and methods for preparing cell samples optimized for dual staining. More particularly, the present invention relates to kits and methods which can be used to recover nucleated cells from bone marrow or peripheral blood samples, which nucleated cells are amenable to a plurality of staining methods and as such can be analyzed using various imaging modalities.

Various cell imaging approaches are routinely utilized for both research and diagnostic purposes. Several cell imaging methods are currently used in clinical and research practice for the diagnosis of hematological malignancies including cancers.

The basic diagnostic tool used in the current practice is a cytological examination of peripheral blood (PB) and bone marrow (BM) cells (J. D. Bauer, Clinical laboratory methods (9^{th} ed.) Mosby, St. Louis, 1982). In this method abnormal frequencies of certain cell types determine the initial diagnosis and classification of hematological malignancies and various kinds of leukemia.

For a more precise diagnosis of leukemia or other hematological malignancies specific markers can be detected using an immunocytochemistry (ICC) assay (Kurec A S et al., 1988, Clin Lab Med; 8: 223-36; Erber W N et al., 1986, Lancet, 1(8484): 761-5).

More recently, tumor cytogenetics and especially Fluorescence In Situ Hybridization (FISH) has contributed to a deeper insight into the chromosomal aberrations characteristics to cancer cells (Kelly L et al., 2002, Curr Opin Oncol, 14: 10-8; Chang S S et al., 1997, Cytobios 90: 7-22). Knowing the precise chromosomal aberration occurring in a certain cancer can contribute to cancer therapy and especially to gene therapy.

For a comprehensive diagnosis of hematological malignancies all the abovementioned diagnostic methods should be employed.

Cell Preparation

The preparation of cells for microscopic evaluation includes two major steps of cell enrichment and fixation to slides. Following is a description of currently utilized cell preparation methodology.

Enrichment

In a blood sample, since the ratio of RBC to nucleated cells is approximately 1000 to 1, respectively, an enrichment step is required before a staining method is employed. Enrichment can be achieved in numerous ways known in the art, including Buffy-Coat, cell lysis, gradient filtering and physical filtering (Ogata K, 2001, Int J Hematol 74: 272-6; Knaust E et al., 2000, Haematologica 85: 124-32; McCarthy D A et al., 1990, J Microsc 158 (Pt 1): 63-72). Each of these methods removes a different type of unwanted cells. For example, cell lysis removes the RBC in the sample, while cell gradients can filter out different cell types (e.g. a gradient at density 1.077 g/cm\hat{ }3 is optimal for lymphocytes separation). This is of particular importance especially in cases where a cell culture is established from one type of cells only. However, for some applications such as differential counts it is crucial to preserve the relative fraction of each cell-sub-population of the blood sample.

Fixation

Blood or bone marrow cells are usually fixed to the microscopic slides. There are several methods of cell fixation known in the arts, including cell smearing, cell dropping and cyto-spinning.

Cell smearing is the most common method used for evaluating the cell morphology. In this method, a drop of body fluid such as blood, bone marrow or sputum is gently spread on the top of a microscopic slide by a flat tool. Since no filtering is employed, the cells in the smear reflect the authentic cell population of the sample and the cell morphology is usually preserved. In addition, cell smearing is fast and easy to perform. However, if the cells of interest are rare as compared with the general cell population in the sample (e.g. blood) it is hard to locate them on the slide. In addition, cells are often not evenly spread over the slide and might overlap and mask other cells. For example, in a blood smear, most of the nucleated cells are obscured by red blood cells.

Cell dropping is a simple method of slide preparation in which the cells are treated prior to their fixation on the slide, diluted in a liquid medium and dropped on the slide from a considerable height (30-50 cm). In this technique the cells are evenly spread and flattened on the slide. The main disadvantage of this method is that pre-processing destroys the cytoplasm and interferes with cell morphology. However, it will be appreciated that when practiced on a blood sample, the pre-processing results in red blood cell lysis thereby enriching the nucleated cell fraction.

Cyto-spinning is a method in which the cells are pretreated, diluted and centrifuged onto a microscopic slide. In this method cell density is controlled, cell cytoplasm is not intensely removed and cells are flattened over the slide by the power of centrifugation. However, due to the forces employed, the cytoplasm are often damaged or distorted which harms the cell morphology.

Cell staining

In order to analyze blood or bone marrow samples the cells have to be stained. Staining methods are classified into specific and non-specific staining.

The non specific staining methods, e.g. Giemsa and Papanicolau, are based on the binding of a chromogen to general DNA or RNA which makes the nucleus and cytoplasm visible for microscopic observation. The morphology of the cells i.e., the cells size, shape and relative size is further evaluated and the cells are identified according to their type.

The specific staining methods are based on binding or activity of specific proteins or DNA contained within the cells.

One of these methods, immunocytochemistry (ICC), is based on the binding of labeled antibodies to antigens present on the cell, making the cell compartment that contains the antigen visible for microscopic evaluation. In order to view all cells in the sample, immunocytochemistry is often accompanied by delicate counterstaining of the cell nuclei and cytoplasm.

Activity staining is another specific staining method based on the enzymatic activity preserved within the cells. To preserve activity, cells should be gently treated and fixed to the slides. To complete the microscopic evaluation it is recommended to counterstain the cells using a chromogen that binds to the cell compartments that do not possess the labeled activity. For example, if the activity is restricted to cell cytoplasm, counterstaining of the cell nucleus is recommended.

For karyotype analysis (i.e., examination of the number, morphology and the appearance of the chromosomes) in order to identify chromosomal aberrations, cells are cultured and chromosomes visualized. Traditional karyotype analysis requires preparation of chromosomes at the metaphase stage of division, which enables ultimate visualization. This is a laborious and time consuming procedure. The FISH technique, enables information on specific chromosomal aberration without the need for cell culturing and metaphase preparation. In this method fluorescent DNA markers are hybridized to specific known chromosomal regions within the cell. The cell cytoplasm and membranes are completely destroyed and the DNA of the chromosomes is denatured prior to hybridization with labeled nucleic acid probes. Fluorescent signals represent single chromosomal markers in a precise and localized way which enables the detection of numerical aberrations, translocations, inversions, duplications and deletions of part of the chromosomes.

Cell Viewing

Cells can be viewed and evaluated under the microscope using a subject's eyes or an automated scanning and image analysis apparatus. For automated scanning, cells should be evenly spread, non-overlapping, and flattened over the slide.

In addition, for accurate image analysis and to avoid false positive and negative results the staining should be as standard as possible so that the interpretation of the different tones of colors would correspond to the actual material contained within the cells. For example, in case of immunocytochemistry (ICC) with antibodies that recognize the B lymphocytes, if the chromogenic reaction that yields the color would occasionally yield a very light color, the machine might interpret it as a negatively stained cell which will add a false negative to the analysis. On the other hand, if the antibody binds non-specifically to other cells a chromogenic reaction would develop on those cells and the scanner will read it as a positively stained cell, i.e., a false positive result.

Cell flattening is especially important for automated cell scanning at higher resolutions. As the imaging resolution increases the depth of the focus decreases. Thus, if the cells are not well-flattened on the slide parts of the cells might become out of focus.

Double Staining

As mentioned above, a comprehensive analysis of hematological malignancies requires several staining methods of the PB or BM samples. Double staining is often needed in cancer patients, especially while in remission, in order to trace the residual cancer cells in the patient's sample.

However, it is often the case that cells prepared for a certain staining method would not be suitable for a second staining method.

For example, fixation of cells by cell dropping may be optimal for FISH analysis but worthless for morphological analysis since the cell cytoplasm is completely destroyed by the pre-treatment. In another case, cell smears are compatible with cell morphology but are not optimal for FISH analysis due to overlapping cells in the slide and the relatively low number of nucleated cells.

In addition, double staining might result in inadequate results due to interference between the two staining methods. For example, the material used for the first staining method might leave some remnants on the cells, which appear as background to the second staining method. On another case the chromogenic substrates used by one staining method might obscure the chromogens used by the second staining method.

As a result of these obstacles the current medical and research practice is to apply one staining method per sample at a time. Thus, a sample stained for morphological analysis by Giemsa stain will not be further analyzed for chromosomal aberrations by FISH analysis.

There is thus a widely recognized need for, and it would be highly advantageous to have, a method of cell preparation that would be suitable for double or triple staining devoid of the above limitations.

The present inventors uncovered methods and kits of cell preparation enabling triple staining of a sample and dual imaging of the chromogenic and fluorescent signals in a way suitable for automated cell scanning.

### SUMMARY OF THE INVENTION

According to one aspect of the present invention there is provided a method of preparing nucleated peripheral blood or bone marrow cells optimized for at least dual mode imaging, the method comprising: (a) isolating nucleated cells from a peripheral blood or a bone marrow sample; and (b) resuspending the nucleated cells in the presence of a morphology preserver including at least 1% serum, and recovering a cell fraction thereby preparing the nucleated peripheral blood or bone marrow cells optimized for at least dual mode imaging.

According to another aspect of the present invention there is provided a method of preparing nucleated blood or bone marrow cells for at least dual mode imaging, the method comprising: (a) isolating nucleated cells from a peripheral blood or a bone marrow sample; and (b) resuspending the nucleated cells in the presence of a morphology preserver including at least 1% serum, and recovering a cell fraction; (c) staining the cells of the recovered cell fraction with at least one stain thereby preparing nucleated peripheral blood or bone marrow cells for dual mode imaging.

According to yet another aspect of the present invention there is provided a method of analyzing a peripheral blood or bone marrow sample, the method comprising: (a) isolating nucleated cells from the peripheral blood or bone marrow sample; and (b) resuspending the nucleated cells in the presence of a morphology preserver including at least 1% serum, and recovering a cell fraction; (c) staining the cells of the recovered cell fraction with at least one stain to thereby obtain stained cells; (d) sequentially and/or simultaneously exposing the stained cells to at least two imaging modes, thereby analyzing the peripheral blood or bone marrow sample.

According to further features in preferred embodiments of the invention described below, step (a) is effected using a density gradient.

According to still further features in the described preferred embodiments the density gradient is a Ficoll based gradient.

According to still further features in the described preferred embodiments the serum is Fetal Calf Serum.

According to still further features in the described preferred embodiments the morphology preserver includes serum at a concentration selected from a range of 1% to 10%.

According to still further features in the described preferred embodiments the morphology preserver includes 5% serum.

According to still further features in the described preferred embodiments the morphology preserver also includes a tissue culture medium.

According to still further features in the described preferred embodiments step (a) is effected by centrifugation of the peripheral blood or the bone marrow sample.

According to still further features in the described preferred embodiments the method further comprising the step of lysing red blood cells of the peripheral blood or the bone marrow sample prior to, and/or following step (b).

According to still further features in the described preferred embodiments the lysing of the red blood cells is effected by subjecting the cell fraction to an hypotonic solution.

According to still further features in the described preferred embodiments the recovering is effected by cytospinning of the cell fraction.

According to still further features in the described preferred embodiments the at least one stain is selected from a group consisting of a morphological stain, an immunological stain, an activity stain and a cytogenetical stain.

According to still further features in the described preferred embodiments the morphological stain is selected from a group consisting of May-Grünwald-Giemsa stain, Giemsa stain, Papanicolau stain and Hematoxyline stain.

According to still further features in the described preferred embodiments the immunological stain is selected from a group consisting of fluorescently labeled immunohistochemistry, radiolabeled immunohistochemistry and immunocytochemistry.

According to still further features in the described preferred embodiments the activity stain is selected from a group consisting of cytochemical stain and substrate binding assays.

According to still further features in the described preferred embodiments the cytogenetical stain is selected from a group consisting of fluorescent in situ hybridization (FISH) stain, radiolabeled in situ hybridization, Digoxygenin labeled in situ hybridization and biotinylated in situ hybridization.

According to still further features in the described preferred embodiments step (d) is effected using an automated cell imaging device.

According to still another aspect of the present invention there is provided a kit for preparing nucleated blood or bone marrow cells for dual mode imaging, the kit comprising a first container including a cell separation reagent suitable for recovering white blood cells from a biological sample and a second container including a morphology preserver including at least 1% serum.

The present invention successfully addresses the shortcomings of the presently known configurations by providing kits and methods useful for preparing cell samples which are optimized for dual staining

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a typical blood sample prepared according to the teaching method of cell preparation described herein Examples. Note the relatively low number of red blood cells (stained in pink) and the abundance of the nucleated cells (stained in purple) in the sample. Magnification is ×20;

FIG. 2 illustrates a typical blood sample prepared according to methods used by prior arts approaches (J. D. Bauer, Clinical laboratory methods (9^{th} ed.). 1982. Mosby, St. Louis). Note the huge number of red blood cells (RBC, stained in brown pink) and the very few nucleated cells (stained in purple, cells marked with “N” and “PMN”) represented in the sample. Magnification is ×20;

FIGS. 3a-b illustrate dual imaging of blood cells labeled by immunocytochemistry (ICC) for CD3 (T-lymphocytes) and CD19 (B-lymphocytes) (FIG. 3a, Magnification ×20) followed by FISH (FIG. 3b, Magnification ×63). The B and T lymphocytes are labeled in brown and pink, respectively, while the X and Y chromosomes are labeled in green and red, respectively. Note that while all the ICC-pink-labeled cells, i.e., the T lymphocytes (FIG. 3a, cells marked with “T”) are labeled following FISH with two green signals, demonstrating the presence of XX chromosomes on their nuclei (FIG. 3b, cells marked with “XX”), the B lymphocyte in the center of the image which is labeled in brown (FIG. 3a, cell marked with “B”) is labeled with green and red signals, demonstrating the presence of X and Y chromosomes in its nuclei (FIG. 3b, cell marked with “XY(1)”). In addition, other ICC-unlabeled cells (FIG. 3a, cells marked with “O”) are showing green and red FISH signals, demonstrating the XY chromosomes in their nuclei (FIG. 3b, cells marked with “XY(2)” and “XY(3)”).

### DESCRIPTION OF THE PREFERRED EMBODIMENTS

The present invention is of methods of nucleated cell preparation from peripheral blood and bone marrow samples which can be utilized for multiple staining optimized for dual mode imaging.

The principles and operation of the methods of cell preparation and staining according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Classification, diagnosis and treatment of hematological malignancies are typically based on cytological examination of peripheral blood and bone marrow cells.

Although morphological analysis provides an important data, for most hematological malignancies it sets up only the first step towards complete diagnosis. Further cytogenetic and/or immunocytochemical analyses are often required for identifying chromosomal aberrations and distinguishing between cells that are morphologically indistinguishable, respectively. For example, T and B lymphocytes are morphologically indistinguishable. Therefore, a cell lineage specific antibody is required for classifying these cells as B or T lymphocytes.

Thus, for a comprehensive diagnosis of hematological malignancies all the abovementioned diagnostic tools should be employed.

Although advantageous, multiple staining of cell samples is not currently practiced since cell preparation methodology which can be used for preparing cells suitable for multiple staining, is not available. In order to stain a single sample with more than one type of stain (e.g., morphological stain and immunostaining), cell preparation must be conducted such that a recovered cell sample is highly amenable to more than one staining procedure since a specific set of conditions used for one staining method are usually inappropriate for use in another staining method.

For example, for FISH analysis, the cells are typically prepared by hypotonic treatment, fixation and cell dropping, a technique which destroys the cell cytoplasm and therefore prevents morphology evaluation of the cells. On the other hand, cell smearing is ideal for cell morphology evaluation but less effective for FISH analysis.

While reducing the present invention to practice, the present inventors have established a unique cell preparation protocol, which retains cell morphology.

As described hereinunder and in the Examples section which follows, the novel cell preparation protocol of the present invention can be efficiently utilized to enrich nucleated cells from peripheral blood and bone marrow samples and allows for the first time to perform highly qualitative multiple staining of cells using a variety of imaging modalities.

Thus, according to one aspect of the present invention there is provided a method of preparing cells of peripheral blood or bone marrow sample, which is optimized for at least dual mode imaging.

The method according to this aspect of the present invention is effected by isolating nucleated cells from a peripheral blood or a bone marrow sample.

Isolated nucleated cells are resuspended in the presence of a morphology preserver, which includes at least 1% serum. As used herein a “morphology preserver” refers to a serum based solution which preserves the morphology of a cellular sample (e.g., nucleated cells) even following application of physical force, such as centrifugal force, thereby optimizing the sample for multiple staining methods and imaging modalities. It is conceivable that the morphology preserver according to this aspect of the present invention mimics the in vivo environment of blood cells. In-vivo blood cells are suspended in plasma. The plasma contains about 92% water, 6-8% proteins as well as salts, lipids and blood sugar, at a PH≅-7.4. Addition of serum to a nutrient solution such as a growth medium enriches the solution with glucose and proteins rendering it similar to blood plasma. Consequently, blood cells suspended in the morphology preserver of the present invention retain morphology even under centrifugal force.

Finally, a cell fraction which includes nucleated peripheral blood or bone marrow cells is recovered. Such cells are optimized for multiple staining and as such can be analyzed using various imaging modalities.

The cell preparation method of the present invention can be carried out using conventional laboratory techniques and equipment.

A blood or bone marrow sample can be collected from a subject using any conventional technique known in the art, such as, for example, venipuncture, usually of the antecubital vein. The volume of blood collected typically ranges between 3-6 ml, but may be more or less according to a need.

Bone marrow samples are more difficult to retrieve but they are often aspirated for clinical diagnosis with a needle from the Sternum or Hip bones. The volume of the bone marrow sample collected is usually 1-3 ml.

The blood or bone marrow samples can be collected into vacuum containers, typically in the presence of one or more anticoagulants, such as, but not limited to, acid-citrate-dextrose (ACD), ethylenediaminetetraacetic acid (EDTA), heparin, and citrate-phosphate-dextrose-adenine (CPDA). The collected blood or bone marrow sample may be stored for up to 4 days at 4° C. Preferably, the blood or bone marrow sample is processed as described below within 24 hours following collection, since longer storage time may damage the morphology. As is mentioned hereinabove, once the blood or bone marrow sample is obtained, nucleated cells are isolated.

Isolation of nucleated cells can be effected by diluting the blood or bone marrow sample in phosphate buffered saline and subjecting the diluted sample to a density gradient (e.g., Ficoll) which separates the nucleated cells from the blood (un-nucleated cells e.g., erythrocytes) or bone marrow sample. The density gradient is subjected to a force such as a centrifugal force, which accelerates the separation process.

When subjected to centrifugation for a predetermined time period, the cells contained in the blood or bone marrow sample migrate through the cell gradient and form an opaque interface. Following this time period, the nucleated cells can be retrieved using, for example, a pipette, transferred into another tube and washed from excess of cell gradient materials with, for example, phosphate buffered saline. Nucleated cells can be further subject to a centrifugation force, which forms a cell pellet. Further description of density gradient isolation of nucleated cells is provided in the Examples section which follows.

The cell pellet formed following the isolation of nucleated cells according to the method described above can be further subjected to red blood lysis procedure in which a hypotonic solution is applied on the cells for a short predetermined time.

Alternatively, nucleated cells can be isolated using other methods known in the art. These include agglutination of red blood cells with phytohemagglutinin (Ehrlich-Kautzky et al., 1991, Biotechniques. 10: 39-40), clumping red blood cells with Methylcellulose (Marchand and Pelletier, 1977, Int J Vitam Nutr Res. 47: 236-47), and utilization of various red blood cell lysis solutions capable of enriching the nucleated cell fraction in a blood or bone marrow sample.

As described hereinabove, nucleated cell pellet are resuspended in a morphology preserver.

The morphology preserver includes serum at a concentration between 1% to 10%, more preferably, 2% to 8%. Most preferably the morphology preserver according to this aspect of the present invention includes 5% serum.

The serum included in the morphology preserver of this aspect of the present invention can be any commercially available serum, provided that it is not contaminated with bacteria and/or viruses. Examples include, but are not limited to, defined fetal bovine serum, characterized fetal bovine serum, standard fetal bovine serum all available from HyClone Inc. (www.hyclone.com).

The serum of the morphology preserver is preferably diluted in a tissue culture medium. Preferred media include but are not limited to Ham's F-10 (commercially available from Hyclone Inc.) and other media suitable for culturing mammalian cells or white blood cells.

Preferably, to obtain qualified images the morphology preserver includes high quality serum and culture medium.

According to a presently preferred embodiment of this aspect of the present invention the morphology preserver includes 5% Fetal Calf serum in a tissue culture medium (e.g., HAM F-10 medium), which facilitates cellular morphology preservation, during cytospinning.

The morphology preserver of the present invention can be easily prepared using conventional mixing and dilution techniques well known to one of skill in the art. Further details of preparation of the morphology preserver are given in the Examples section which follows.

Once protected against physical force (by the morphology preserver) nucleated cell fraction is recovered. Recovery can be effected using various techniques known to those of skill in the art such as by cytospinning.

Cytospinning according to the present invention is effected by placing the isolated nucleated cells in a container placed on the top of a microscopic slide. The container includes a small hole through which the cells, when subject to a centrifugational force, can migrate sediment onto the slide, while in appropriate cocentration creating a monolayer of cells. Following cytospinning the slides are dried in a horizontal position for a predetermined time.

As described in the Examples section which follows, the nucleated cells placed in the container are preferably at a density of about 1,000 cell/mm².

Cytospinning according to the present invention can be performed using any cytospin device known in the arts and the amount and concentration of cells placed in the container would be determined according to manufacturer's instructions.

Thus, the method according to this aspect of the present invention provides a novel approach for recovering a blood or bone marrow sample cell fraction which is highly amenable to multiple staining procedures and thus can be stained and viewed using a variety of stain types.

Following is a non-limiting description of a number of staining procedures, which can be effectively applied on the nucleated cell-preparation, obtained according to the teachings of the present invention, described hereinabove.

Morphological Staining

Morphological stains bind non-specifically to cell compartments rendering them visible for microscopic observation. Examples, include but are not limited to May-Grünwald-Giemsa stain, Giemsa stain, Papanicolau stain and Hematoxyline stain.

Morphological staining can be effected by simple mixing, diluting and washing laboratory techniques and equipment. Following the application of the appropriate stain, the microscopic slides containing stained cells can viewed under a microscope equipped with either a bright or a dark field source of light with the appropriate filters according to manufacturer's instructions.

Immunological Staining

Immunological staining is based on the binding of labeled antibodies to antigens present on or within the cells. Examples of immunological staining procedures include but are not limited to, fluorescently labeled immunohistochemistry, radiolabeled immunohistochemistry and immunocytochemistry.

Immunological staining is preferably followed by counterstaining the cells with a dye which binds to non-stained cell compartments. For example, if the labeled antibodies bind to antigens present on the cell cytoplasm, a nuclear stain (e.g., Hematoxyline stain) is an appropriate counterstaining.

Antibody labeling can be effected using numerous labeling modes known in the art.

For example, antibodies can be conjugated to a fluorescent dye (e.g. fluorescent immunohistochemistry) in which case visualization is direct using a fluorescent microscope.

Antibodies can also be radiolabeled with certain isotopes, in which case bound antibodies are retrieved following the development of a photographic emulsion which results in localized silver grains in cells containing bound antibodies. These silver grains can be further viewed under a light microscope.

Alternatively, antibodies can be conjugated to an enzyme (e.g., horseradish peroxidase (HRP)) in which case, upon binding to a chromogenic substrate specific to the conjugated enzyme, the enzyme catalyzes a reaction in which the chromogenic substrate becomes detectable when viewed under a light or a fluorescent microscope.

Activity Staining

According to this method, a chromogenic substrate is applied on the cells containing an active enzyme. The enzyme catalyzes a reaction in which the substrate is decomposed to produce a chromogenic product visible by a light or a fluorescent microscope. Examples of commonly practiced activity staining procedures include but are not limited to cytochemical stain and substrate binding assays.

Activity staining also include substrate binding assays which utilize endogenous substrates in order to activate a chromogenic dye bound to an ectopically introduced enzyme. In this method, once the enzyme binds to its natural substrate on the cell, a conformational change within the enzyme molecule activates the conjugated dye in such a way that a chromogenic product will deposit on the cell. The chromogenic product can be further viewed under a light of a fluorescent microscope.

Cytogenetical Staining

Useful for identification of specific chromosomal aberrations. Examples of cytogenetical stainings include but are not limited to fluorescent in situ hybridization (FISH), radiolabeled in situ hybridization, Digoxygenin labeled in situ hybridization and biotinylated in situ hybridization.

Numerous nucleic acid labeling techniques are known in the art. For example, a fluorescent dye can be covalently attached to either the 5′ or 3′ end of a nucleic acid probe. Following hybridization, the labeled probe can be directly retrieved using a fluorescent microscope.

Alternatively, a nucleic acid probe can be directly labeled with a radioactively labeled nucleotide such as ³⁵S-ATP. In this case the labeled nucleotide can be incorporated to the nucleic acid probe by conventional labeling techniques known to those skilled in the art of molecular biology. Labeling techniques used by the present invention include, but not limited by, Nick Translation, Random Primed Labeling, End Labeling with a polynucleotide kinase etc. Following hybridization, the labeled nucleic acid probes are retrieved by the development of a photographic emulsion which produces dark silver grains that can be further viewed under a light microscope.

Optionally, a nucleic acid probe can be prepared by incorporating a Digoxygenin (DIG) labeled nucleotide to the nucleic acid probe. Digoxygenin labeled nucleotides are prepared according to the labeling techniques described herein above. Following hybridization, an anti-DIG antibody is applied on the cells. Anti-DIG antibodies can be directly labeled with a fluorescent dye in which case the hybridization signal is viewed under a fluorescent microscope or they can be conjugated to an enzyme (e.g., HRP), in which case upon the addition of a chromogenic substrate will produce a color that can be further viewed under a light or a fluorescent microscope.

The nucleic acid probes of the present invention can be also conjugated to a biotin molecule at the 5′ or 3′ end of the nucleic acid probe. In this case, following hybridzation, and an avidin or a streptavidin molecule is further applied on the cells. The avidin or streptavidin molecules used by the present invention can be directly labeled with a fluorescent dye or can be conjugated to an enzyme which will further produce a chromogenic product once the appropriate substrate is employed.

It is well appreciated that for comprehensive diagnosis of hematological malignancies numerous diagnostic methods should be applied. However, until today multiple analyses of nucleated cells have been impossible to qualitatively perform. For example, hypotonic treatment followed by fixation of cells and cell dropping on the slide is compatible with FISH analysis but worthless for morphological analysis.

Because of their well preserved morphology, stained peripheral blood or bone marrow samples prepared as described above can be sequentially and/or simultaneously exposed to at least two imaging modes (i.e., dual imaging), to thereby phenotype information.

A dual imaging is for example when a first image is obtained following immunocytochemistry (ICC) or morphology staining and a second image is obtained following a FISH analysis. This enables the user to correlate a readout obtained by one staining method (e.g., ICC) to a readout obtained by another staining method (e.g., FISH). An example of a dual imaging is provided in the Examples section which follows. Briefly, following ICC and FISH analysis, the T lymphocytes in a male patient blood sample were shown to originate from a female donor of a bone marrow transplant as they included female sex chromosomes. In comparison, the B lymphocytes in the same blood sample originated from the male host's bone marrow since they exhibited the signals of both X and Y chromosomes, as expected from a male cell.

Imaging can be effected using a cell imaging device (e.g., microscope). Preferably an automated imaging device which is capable of integrating a number of signals and execute multiple analyses simultaneously is used. An example for an automated cell imaging device is the Duet™ (Bio View, Israel) disclosed in PCT/IL00/00101.

Thus, the methods of the present invention can increase the information which can be obtained from a single blood or bone marrow sample and improve the accuracy diagnosis.

The methods of the present invention can be used in various clinical and research applications. For example, in diagnosis and phenotyping of hematological cancers, autoimmune diseases such as systemic lupus erythematosus and systemic sclerosis (Migliore et al., Mutagenesis (1999), 14: 227-31), and various inherited diseases such as Wiskott-Aldrich, which are caused by chromosomal aberrations (Lutskiy et al., Hum Genet (2002), 110: 515-9).

It will be appreciated that the hereinabove described reagents can be included in a diagnostic kit. For example a kit for preparing nucleated blood or bone marrow cells suitable for dual mode imaging, can include a morphology preserver packaged in a one container and a cell separation reagent (e.g., Ficoll based gradient density solution) packaged in a second container with appropriate buffers and preservatives and used for diagnosis or for directing therapeutic treatment.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

Preparation of Cytospin Slides of Peripheral Blood and Bone Marrow Cells

Sample dilution: Peripheral blood (PB) or bone marrow (BM) cells are diluted with equal volume (up to 3 ml) of wash solution (Bio View Cat. # BV-000-05).

White blood cells (WBC) separation: Six ml of a Ficoll-based density gradient WBC separation reagent (Bio View Cat. # BV-000-09) are poured into a 15 ml culture tube (Corning, N.Y., USA). The diluted PB or BM sample is then carefully layered over the WBC separation reagent and the tubes are centrifuged for 30 minutes at 400×g at room temperature (20-25° C.). Following centrifugation, the upper layer is carefully removed with a Pasteur pipette up to a distance of 0.5 cm from the opaque interface containing the white blood cells. The opaque interface is transferred with a Pasteur pipette into a clean 15 ml conical test tube (Falcon, N.J., USA) and is gently mixed with 5 ml of the wash buffer. The cells are then centrifuged for 10 minutes at 250×g at room temperature. The supernatant is aspirated and discarded and the white pellet is retained; the wash procedure is repeated three times. If the pellet appears to be mixed with red cells, an RBC lysis procedure such as that described below is employed before the second wash.

RBC lysis procedure: To reduce the number of RBC in the WBC pellet 0.5 ml of RBC lysis reagent (Bio View Cat. # BV-000-12) is added to the WBC pellet and mixed by gentle aspiration. Following 10 seconds of incubation the solution is neutralized with 0.5 ml of Neutralization buffer (Bio View Cat. # BV-000-13). The cells are further centrifuged for 10 minutes at 250×g at room temperature.

Preparation of cytospins: For cell suspension the WBC pellet is resuspended with 300 μl of Morphology preserver reagent (Bio View Cat. # BV-000-03). The concentration of the WBC suspension is determined using a counting chamber device (Improved; 0.0025 mm², Neubauer, Germany). Cells are further diluted to an optimal concentration of 1,000 cells/mm² and placed in a cytocentrifuge (Kubota, Japan) for centrifugation according to manufacturer's instructions. Slides are then dried in a horizontal position at room temperature overnight.

Morphology staining: For morphological observations, slides are stained with May-Grünwald-Giemsa which labels the nucleus in deep purple and the cytoplasm in various shades from pink to blue. Slides are dipped in May-Grünwald (Cat. # MAY-1, Sigma, USA) stain for 2 minutes and briefly rinsed with distilled water. Slides are then dipped in a diluted (1:20 in distilled water) Giemsa stain (Cat. # GS-500, Sigma, USA) for 7 minutes, rinsed under tap water and air-dried.

Immunohistochemistry: An immunocytochemistry (ICC) staining assay is employed to detect specific proteins contained within the cells. To prevent background signals, slides are first blocked at room temperature for 30 minutes by applying a blocking reagent (Bio View Cat. # BV-020-08). After tapping off the excess blocking reagent, the slides are incubated for 30 minutes with a diluted primary antibody (1:5-1:5000). The antibody bound slides are then washed twice for 5 minutes, with a wash solution (Bio View Cat. # BV-020-05). A secondary HRP-conjugated antibody (BioView cat. # BV-020-10) is then applied to the slides which are incubated for 30 minutes at room temperature. The slides are then washed twice for 5 minutes with the wash solution and a chromogenic substrate (BioView cat. # BV-020-11) is then added to the slides which are subsequently incubated for 15 minutes and rinsed gently in distilled water.

Hematoxylin counterstain: Following ICC staining, slides can be further stained with Hematoxylin (Sigma, USA) which labels the nuclei and enables morphological evaluation. Slides are immersed in an aqueous solution of Hematoxylin for 5-20 minutes, rinsed in distilled water and further washed under running tap water until a blue color is detected.

ICC and morphology observation: When viewed under a light microscope, cells treated with the ICC and Hematoxylin staining procedures described above, display a blue stained nucleus and a red (ICC-positive cells) or pink (ICC-negative cells) stained cytoplasm.

Fluorescent In Situ Hybridization (FISH): Following ICC and May-Grünwald-Giemsa staining, slides can be further subjected to FISH analysis. For distaining and fixation, slides are immersed for one hour in an ice-cold methanol/acetic acid fixative (prepared in a 3:1 ratio, respectively) and rinsed twice in phosphate buffered saline (PBS) for 2 minutes at room temperature. For digestion, slides are dipped for 5 minutes at 37° C. in an already warmed HCl solution containing a digestion enzyme (25 μl of a digestion enzyme solution (Bio View BV-010-06) in a 50 ml solution of 10 mM HCl). Slides are rinsed twice in PBS for 5 minutes and dehydrated for 2 minutes in a series of ice-cold 70%, 80% and 100% ethanol and dried on a 37° C. hot plate for 5 minutes. The FISH probe is denatured for 5 minutes at 75° C., applied on the slides, covered with a rubber cement sealed coverslip. Slides are further denatured for 5 minutes with the probe at 75° C. Hybridization is performed according to probe's manufacturer's instructions. Following hybridization, coverslips are removed and slides are rinsed in a 0.4×SSC solution (sodium chloride/sodium citrate, 60 mM/6 mM, respectively) at 73° C. for 5 minutes. For a complete removal of excess of probes from slides they are further washed for 2 minutes at room temperature in a 2×SSC/NP-40 solution (sodium chloride/sodium citrate/NP-40, 300 mM/30 mM/0.01%, respectively). After tapping off the excess wash solution, the Blue View counterstain (Bio View Cat. # BV-010-07) is employed. When viewed under a fluorescent microscope, cells treated with the FISH protocol described hereinabove display light blue stained nucleus and fluorescent signals such as green and red for stained chromosomes.

Results

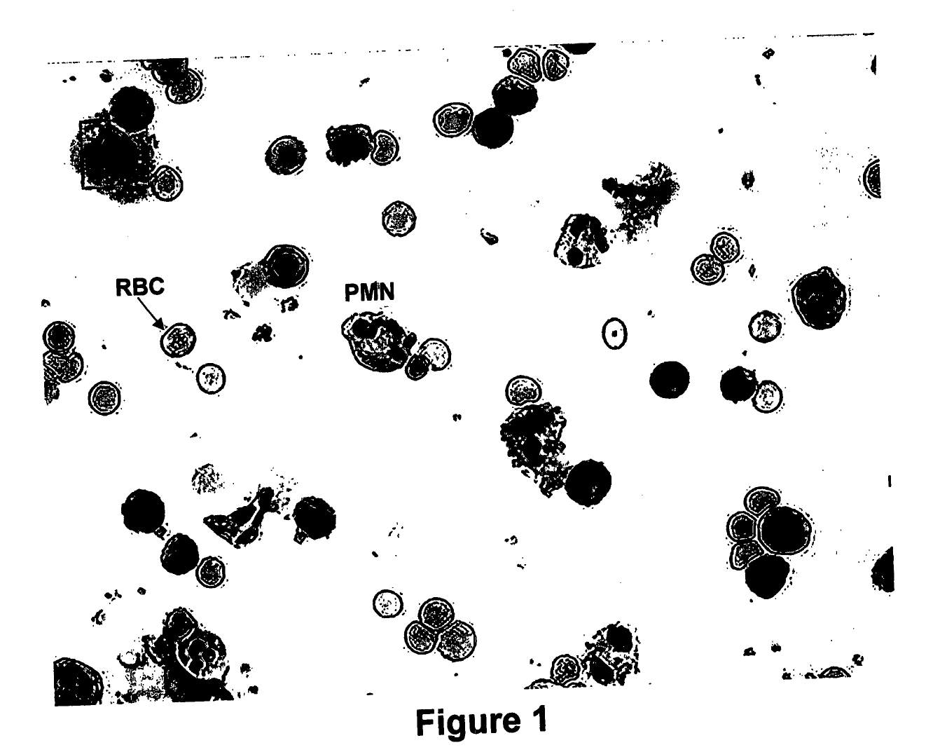

Evaluating blood cells using May-Grünwald-Giemsa staining: In order to analyze the normality of a blood sample, blood cells were processed using the above described cell preparation methodology. Slides containing the processed blood samples were stained with May-Grünwald-Giemsa and been photographed using a Duet™ workstation (BioView Ltd., Israel). Multiple nucleated cells were detected in the sample (FIG. 1, purple cells) in addition to a reasonable number of un-nucleated red blood cells (FIG. 1, pink cells).

Comparing the present methodology to prior art approaches: The blood sample prepared using the method of the present invention was compared to a blood sample from the same source which was prepared using a prior art approach (Carr, J. H. and Rodak, B. F., Clinical hematology Atlas, W. B. Saunders company, Philadelphia, USA, 1999). Slides containing the processed blood samples were stained with May-Grünwald-Giemsa and cells were photographed as described above.

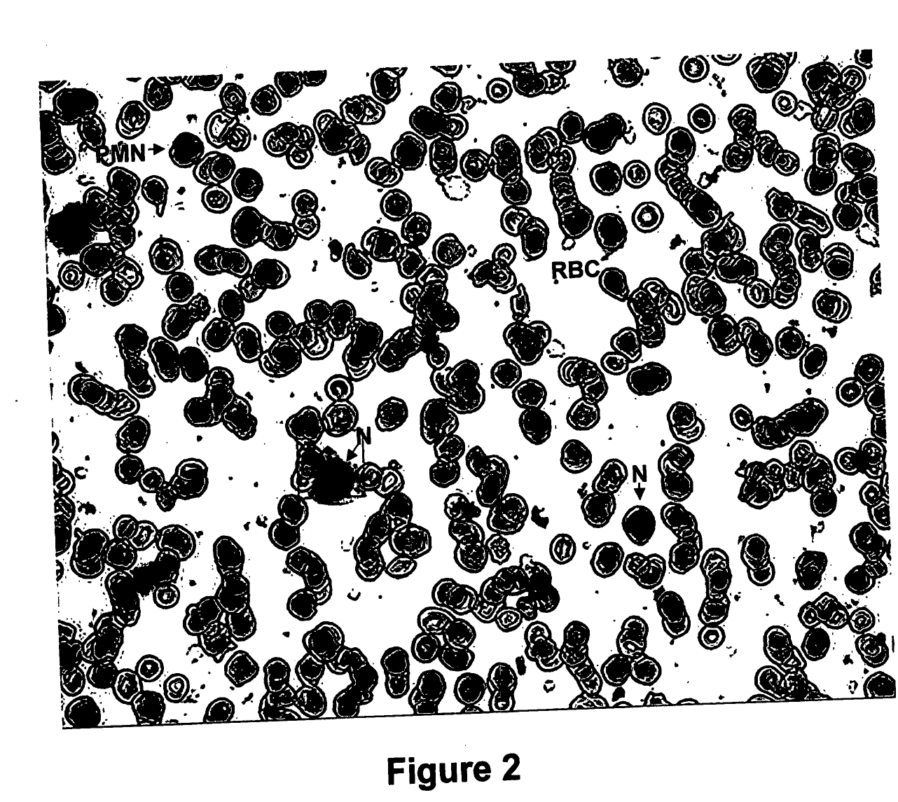

FIG. 2, presents the results obtained for the blood sample prepared using the prior art approach. While multiple nucleated cells (FIG. 1) are present in the blood sample prepared according to the method of the present invention, only three nucleated cells (FIG. 2, cells stained in purple) and multiple un-nucleated RBC cells (FIG. 2, cells stained in light brown) are present in the blood sample prepared according to the prior art approach. In addition, while the polymorphic nuclear cell observed in the blood sample prepared according to the method of the present invention is huge and consists of an amorphic nucleus (FIG. 1, cell marked with “PMN”) the polymorphic nuclear of the blood sample prepared according to the prior art approach is round and relatively small (FIG. 2, cell marked with “PMN”). These results clearly demonstrate the advantage of preparing blood sample cells according to the method of the present invention as compared with other methods known in the arts.

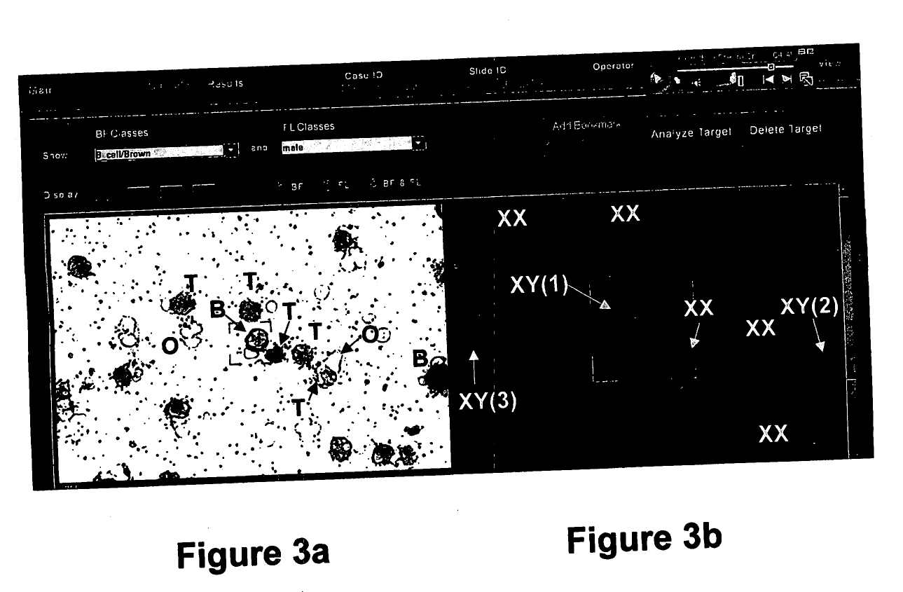

Immunocytochemistry (ICC) of samples prepared according to the teachings of the present invention enables to distinguish between B and T lymphocytes: In a morphologically stained blood sample the B and T lymphocytes are indistinguishable. In order to distinguish between these two cell types, antibodies against specific antigens (CD19 for the B cells, CD3 for the T cells) were applied on a blood sample from an immunodeficiency bone marrow male patient that received a bone marrow transplant from a female donor. The antibody against B cells labels the cells in dark brown (FIG. 3a, cells marked with “B”), while the antibody against T cells labels the cells in pink (FIG. 3a, cells marked with “T”). The ICC results were recorded using the Duet™ imaging apparatus (BioView Ltd., Israel). These results demonstrate that cells prepared according to the method of the present invention are suitable for double immunocytochemistry staining.

Fluorescent In Situ Hybridization (FISH) analysis distinguishes between “self” and “donor” cells: To further distinguish between the “host” male cells and the “donor” female cells a FISH analysis according to the method described hereinabove has been further employed on the same blood sample. The FISH probes used for the identification of the X and Y chromosomes were the Vysis (USA) probes Spectrum Green™ Spectrum Orange™, respectively. While the cells originated from the immunodeficiency male patient were labeled with green and red signals representing the X and Y chromosome, respectively (FIG. 3b), the cells originated from the female donor were labeled with two green signals (FIG. 3b). It is noteworthy that all cells subject to FISH analysis are in a simultaneous focus as a result of the cell flattening employed by the method of the present invention. The FISH data have been recorded using the triple filters (DAPI/Orange/Green) on the fluorescent microscope. These results demonstrate that cells prepared according to the method of the present invention are suitable for a successful FISH analysis, which follows an ICC assay on the same sample.

Simultaneous imaging of ICC and FISH analyses: In order to distinguish between “host” and “donor” B and T lymphoblasts, the images obtained from the ICC and FISH analyses were viewed simultaneously using the Duet™ imaging apparatus (BioView Ltd., Israel) with bright field for the ICC and morphology staining and with the triple filter of the fluorescent microscope for the FISH analysis (FIG. 3a,b). These images clearly demonstrate that all positive B cells are of male origin and all T cells are of female origin, i.e., originated from the “donor” bone marrow. These results demonstrate the advantage of dual imaging of ICC and FISH analyses for monitoring bone marrow transplants.

Thus, the unique blood cell processing approach of the present methodology enables one of ordinary skill in the art to extract previously unobtainable information from a single multistained blood sample since the cells are prepared and fixed in a way that is suitable for an ICC assay followed by FISH analysis. As is illustrated hereinabove, these features of the present invention cannot be provided using prior art approaches.

### EXAMPLE 2

### A Kit for Cell Preparation of Cyto-Spin Slides of Blood or Bone Marrow Samples

The following kit (Table 1) is designed for the preparation of cyto-spin slides for the evaluation of the cells' morphology and for further use of the same slides for ICC and FISH assays. Slides can be scanned with the automated scanning system described in PCT/IL00/00101.

TABLE 1
Kit for preparing cyto-spin slides of blood or bone marrow samples

| | | Reagents required for | | |
|---|---|---|---|---|
| | | preparation of kit's | Instructions for | |
| Kit's | BioView | component (including | preparation of kit's | Special |
| components | Ltd Cat. # | supplier's Cat. #) | component | Notifications |
| Wash | BV-000-05 | 20 X PBS (Cat. # I 291, | Diluted 1:20 in | Work in a sterile |
| Solution | | Savyon, Israel) | double distilled | environment |
| | | | water | |
| WBC | BV-000-09 | Histopaque 1.077 (Cat. # | 250 ml of | Work in a sterile |
| Separation | | 1077-1, Sigma, USA); | Histopaque 1.077 | tent. |
| Reagent | | Histopaque 1.119 (Cat. # | are mixed with 750 ml | |
| | | 1119-1, Sigma, USA) | of Histopaque | |
| | | | 1.119. | |
| RBC lysis | BV-000-12 | Water, tissue culture grade | Sodium Azide | Work in a sterile |
| Reagent | | (Cat. # 03-005-1, | 0.1 gr) is added to | environment |
| | | Biological Industries, | 100 ml water | |
| | | Israel) | | |
| Neutralizing | BV-000-13 | NaCl (Cat. # S 7653, | NaCl (3.5 gr) is | |
| Buffer | | Sigma, USA) | mixed in 100 ml | |
| | | | double distilled | |
| | | | water and Sodium | |
| | | | Azide (0.1 gr) is | |
| | | | added. | |
| Morphology | BV-000-03 | F-10 (HAM medium) (Cat. | etal Calf Serum (5 | Work in a sterile |
| preserver | | # 01-090-1, Biological | 1) is mixed with | tent and use a |
| | | Industries, Israel); | AM F-10 (95 ml) and | membrane for |
| | | Fetal Calf Serum (Cat. # | odium Azide (0.1 gr) | filtration. |
| | | 04-001-1, Biological | s added. | |
| | | Industries, Israel). | | |

### EXAMPLE 3

### A Kit for an Immunohistochemistry Assay on Slides

The following kit (Table 2) is designed for an immunocytochemistry (ICC) assay. Following ICC slides can be scanned with the automated scanning system described herein above.

TABLE 2
A kit for Immunohistochemistry assay

| | | Reagents required for | | |
|---|---|---|---|---|
| | | preparation of kit's | Instructions for | |
| | BioView Ltd. | component (including | preparation of | |
| [symbol] it's components | Cat. # | supplier's Cat. #) | kit's component | [symbol] pecial notifications |
| [symbol] ash solution | BV-020-05 | TBS (Cat. # T6664, | TBS powder is | — |
| | | Sigma, USA) | mixed in 1 liter of | |
| | | | distilled water. | |
| [symbol] locking reagent | BV-020-08 | TBS (Cat. # T6664, | TBS is prepared | [symbol] ork in a sterile |
| | | Sigma, USA) | as mentioned | [symbol] nvironment and use a |
| | | Normal goat serum (Cat. | herein above. | [symbol] embrane for |
| | | # 005-000-121, Jackson | Goat serum | [symbol] iltration |
| | | USA) | (freeze-dried) is | |
| | | | reconstituted with | |
| | | | 10 ml of water at | |
| | | | room temperature | |
| | | | for 2 hours. | |
| | | | Reconstitute goat | |
| | | | serum (10 ml) is | |
| | | | mixed with TBS | |
| | | | (90 ml) and | |
| | | | Sodium Azide | |
| | | | (0.1 gr) is added. | |
| [symbol] ntibody diluent | BV-020-04 | TBS (Cat. # T6664, | TBS and goat | [symbol] ork in a sterile. |
| | | Sigma, USA) | serum as prepared | [symbol] nvironment and use a |
| | | Normal goat serum (Cat. | as mentioned | [symbol] embrane for |
| | | # 005-000-121, Jackson | herein above. | [symbol] iltration |
| | | USA) | Reconstitute goat | |
| | | | serum (10 ml) is | |
| | | | mixed with TBS | |
| | | | (90 ml) and | |
| | | | Sodium Azide | |
| | | | (0.1 gr) is added. | |
| [symbol] econdary | BV-020-10 | HRP-goat anti rabbit and | | |
| [symbol] ntibody (HRP | | goat anti mouse (Cat. # | | |
| [symbol] onjugate) | | K 5007, DAKO, USA); | | |
| [symbol] EC substrate | BV-020-11 | 3-amino-9- | | |
| | | ethylcarbazole (AEC) | | |
| | | containing hydrogen | | |
| | | peroxidase (Cat. # K | | |
| | | 3461, DAKO, USA) | | |

### EXAMPLE 4

### A Kit for FISH Analysis

The following kit (detailed in Table 3) is designed to determine cell karyotypes and identify chromosomal aberrations in samples already stained with ICC and/or morphology staining. The FISH data obtained using the following kit are suitable for automated scanning.

TABLE 3
A kit for FISH analysis

| | | Reagents required for | | |
|---|---|---|---|---|
| | | preparation of kit's | Instructions for | |
| | BioView | component (including | preparation of | |
| [symbol] it's components | Ltd. Cat. # | supplier's Cat. #) | kit's component | Special notifications |
| [symbol] igestion | BV-010-06 | Pepsin (Cat. # | HCl (10 μl) are | Enzyme solution is |
| [symbol] nzyme | | 1.07185.01000, Merck, | mixed with | prepared in a |
| | | Germany); | distilled water (10 ml). | seperate container; |
| | | HCl (Cat. # 30721, Sigma, | Pepsin (1 gr) | pH of the digestion |
| | | USA) | is added to the | solution is 3.5 |
| | | | HCl solution and | |
| | | | mixed well. | |
| [symbol] lueView | BV-010-07 | Vectashield (Cat. # H-1000, | Vectrashield with | Product is kept in a |
| [symbol] ounterstain | | Vector, USA); | DAPI (1 ml) is | light protected box at |
| | | Vectashield with DAPI | mixed with | 4° C. |
| | | (Cat. # H-1200, Vector, | Vectrashield (9 ml). | |
| | | USA) | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of preparing nucleated peripheral blood or bone marrow cells optimized for at least dual mode imaging, the method comprising:
(a) isolating nucleated cells from a peripheral blood or a bone marrow sample; and
(b) resuspending said nucleated cells in the presence of a morphology preserver including at least 1% serum, and recovering a cell fraction thereby preparing the nucleated peripheral blood or bone marrow cells optimized for at least dual mode imaging.

2. The method of claim 1, wherein step (a) is effected using a density gradient.

3. The method of claim 2, wherein said density gradient is a Ficoll based gradient.

4. The method of claim 1, wherein said serum is Fetal Calf Serum.

5. The method of claim 1, wherein said morphology preserver includes serum at a concentration selected from a range of 1% to 10%.

6. The method of claim 1, wherein said morphology preserver includes 5% serum.

7. The method of claim 1, wherein said morphology preserver also includes a tissue culture medium.

8. The method of claim 1, wherein step (a) is effected by centrifugation of said peripheral blood or said bone marrow sample.

9. The method of claim 1, further comprising the step of lysing red blood cells of said peripheral blood or said bone marrow sample prior to, and/or following step (b).

10. The method of claim 9, wherein said lysing of said red blood cells is effected by subjecting said cell fraction to an hypotonic solution.

11. The method of claim 1, wherein step (b) is effected via cytospinning.

12. A method of preparing nucleated blood or bone marrow cells for at least dual mode imaging, the method comprising:
(a) isolating nucleated cells from a peripheral blood or a bone marrow sample; and
(b) resuspending said nucleated cells in the presence of a morphology preserver including at least 1% serum, and recovering a cell fraction;
(c) staining said cells of the recovered cell fraction with at least one stain thereby preparing nucleated peripheral blood or bone marrow cells for dual mode imaging.

13. The method of claim 12, wherein step (a) is effected using a density gradient.

14. The method of claim 13, wherein said density gradient is a Ficoll based gradient.

15. The method of claim 12, wherein said serum is Fetal Calf Serum.

16. The method of claim 12, wherein said morphology preserver includes serum at a concentration selected from a range of 1% to 10%.

17. The method of claim 12, wherein said morphology preserver includes 5% serum.

18. The method of claim 12, wherein said morphology preserver also includes a tissue culture medium.

19. The method of claim 12, wherein step (a) is effected by centrifugation of said peripheral blood or said bone marrow sample.

20. The method of claim 12, further comprising the step of lysing red blood cells of said peripheral blood or said bone marrow sample prior to, and/or following step (b).

21. The method of claim 20, wherein said lysing of said red blood cells is effected by subjecting said cell fraction to an hypotonic solution.

22. The method of claim 12, wherein said recovering is effected by cytospinning of said cell fraction.

23. The method of claim 12, wherein said at least one stain is selected from a group consisting of a morphological stain, an immunological stain, an activity stain and a cytogenetical stain.

24. The method of claim 23, wherein said morphological stain is selected from a group consisting of May-Grünwald-Giemsa stain, Giemsa stain, Papanicolau stain and Hematoxyline stain.

25. The method of claim 23, wherein, said immunological stain is selected from a group consisting of fluorescently labeled immunohistochemistry, radiolabeled immunohistochemistry and immunocytochemistry.

26. The method of claim 23, wherein said activity stain is selected from a group consisting of cytochemical stain and substrate binding assays.

27. The method of claim 23, wherein said cytogenetical stain is selected from a group consisting of fluorescent in situ hybridization (FISH) stain, radiolabeled in situ hybridization, Digoxygenin labeled in situ hybridization and biotinylated in situ hybridization.

28. A method of analyzing a peripheral blood or bone marrow sample, the method comprising:
(a) isolating nucleated cells from the peripheral blood or bone marrow sample; and
(b) resuspending said nucleated cells in the presence of a morphology preserver including at least 1% serum, and recovering a cell fraction;
(c) staining said cells of the recovered cell fraction with at least one stain to thereby obtain stained cells;
(d) sequentially and/or simultaneously exposing said stained cells to at least two imaging modes, thereby analyzing the peripheral blood or bone marrow sample.

29. The method of claim 28, wherein step (a) is effected using a density gradient.

30. The method of claim 29, wherein said density gradient is a Ficoll based gradient.

31. The method of claim 28, wherein said serum is Fetal Calf Serum.

32. The method of claim 28, wherein said morphology preserver includes serum at a concentration selected from a range of 1% to 10%.

33. The method of claim 28, wherein said morphology preserver includes 5% serum.

34. The method of claim 28, wherein said morphology preserver also includes a tissue culture medium.

35. The method of claim 28, wherein step (a) is effected by centrifugation of said peripheral blood or said bone marrow sample.

36. The method of claim 28, further comprising the step of lysing red blood cells of said peripheral blood or said bone marrow sample prior to, and/or following step (b).

37. The method of claim 36, wherein said lysing of said red blood cells is effected by subjecting said cell fraction to an hypotonic solution.

38. The method of claim 28, wherein said recovering is effected by cytospinning of said cell fraction.

39. The method of claim 28, wherein said at least one stain is selected from a group consisting of a morphological stain, an immunological stain, an activity stain and a cytogenetical stain.

40. The method of claim 39, wherein said morphological stain is selected from a group consisting of May-Grünwald-Giemsa stain, Giemsa stain, Papanicolau stain and Hematoxyline stain.

41. The method of claim 39, wherein, said immunological stain is selected from a group consisting of fluorescently labeled immunohistochemistry, radiolabeled immunohistochemistry and immunocytochemistry.

42. The method of claim 39, wherein said activity stain is selected from a group consisting of cytochemical stain and substrate binding assays.

43. The method of claim 39, wherein said cytogenetical stain is selected from a group consisting of fluorescent in situ hybridization (FISH) stain, radiolabeled in situ hybridization, Digoxygenin labeled in situ hybridization and biotinylated in situ hybridization.

44. The method of claim 28, wherein step (d) is effected using an automated cell imaging device.

45. A kit for preparing nucleated blood or bone marrow cells for dual mode imaging, the kit comprising a first container including a cell separation reagent suitable for recovering white blood cells from a biological sample and a second container including a morphology preserver including at least 1% serum.

46. The kit of claim 45, further comprising at least one additional container including a stain selected from the group consisting of morphological stain, an immunological stain, an activity stain and a cytogenetical stain.

47. The kit of claim 45, wherein said cell separation reagent is a density gradient.

48. The kit of claim 47, wherein said density gradient is a Ficoll based gradient.

49. The kit of claim 45, wherein said serum is Fetal Calf Serum.

50. The kit of claim 45, wherein said morphology preserver includes serum at a concentration selected from a range of 1% to 10%.

51. The kit of claim 45, wherein said morphology preserver includes serum at a concentration of 5%.

52. The kit of claim 45, wherein said morphology preserver also includes a tissue culture medium.

53. The kit of claim 46, wherein said morphological stain is selected from a group consisting of May-Grünwald-Giemsa stain, Giemsa stain, Papanicolau stain and Hematoxyline stain.

54. The kit of claim 46, wherein, said immunological stain is selected from a group consisting of fluorescently labeled immunohistochemistry, radiolabeled immunohistochemistry and immunocytochemistry.

55. The kit of claim 46, wherein said activity stain is selected from a group consisting of cytochemical stain and substrate binding assays.

56. The kit of claim 46, wherein said cytogenetical stain is selected from a group consisting of fluorescent in situ hybridization (FISH) stain, radiolabeled in situ hybridization, Digoxygenin labeled in situ hybridization and biotinylated in situ hybridization.
